(19) United States
(12) Patent Application Publication
(10) Pub. No.: US 2010/0173822 A1
(43) Pub. Date: Jul. 8, 2010

(54) COMPOSITION COMPRISING SACCARIDES & PEPTIDES & USES THEREOF

(75) Inventors: Huaying Zhang, Beijing (CN); Lisa Yu Zhou, Shanghai (CN); Libo Geng, Shanghai (CN); Muqing Yi, Beijing (CN); Lili Zhou, Beijing (CN)

Correspondence Address: THE COCA-COLA COMPANY;PATENT & TECHNOLOGY DEPT--NAT 19, P. O. BOX 1734, ATLANTA, GA, 30301, US

(73) Assignee: The Coca-Cola Company, Atlanta (US)

(21) Appl. No.: 12/521,170

(22) PCT Filed: Dec. 26, 2007
(86) PCT No.: PCT/US07/26325

§ 371 Date: Mar. 12, 2010

(30) Foreign Application Priority Data
Dec. 30, 2006 (CN) 200610156176.1

(51) Int. Cl. A61K 38/00

(52) U.S. Cl. 514/2

(57) ABSTRACT

The present invention relates to a composition comprising saccharides and peptides in unique proportion for improving the endurance capability of the human body in motion, promoting recovery after motion, reducing body fat, and alleviating fatigue of central nerves during motion. The composition can further comprise vitamins, minerals, flavoring materials and electrolytes. The composition can be a liquid beverage, a solid beverage, a food additive or an energy bar.

### CROSS-REFERENCE TO RELATED APPLICATIONS

This application claims the benefit of Chinese Patent Application Serial No. 200610156176.1, filed on Dec. 30, 2006.

### TECHNICAL FIELD

The present invention relates to a composition comprising saccharides and peptides in unique proportion for improving the endurance capability of the human body in motion, promoting recovery after motion, reducing body fat, and alleviating fatigue of central nerves during motion.

### BACKGROUND ART

During human body motion, such as during sport exercises, energy is mainly obtained from fats and saccharides stored in the body. The storage amount of fats is far more than that of saccharides.

Since the storage amount of fats is far more than that of saccharides, when the human body is subjected to a long period (more than 60 minutes) of motion, the oxidation of fats in vivo will be accelerated in order to maintain human body motion after glycogens in the body are depleted. Thus, supplementing saccharides and enhancing fat mobilization and oxidation in optimal ways is very important for improving the endurance of the human body in motion.

In the meantime, the consumption of a large amount of branched amino acids during human body motion results in fatigue of central nerves.

Fallowfield et al. reported that the supplementation of a beverage comprising saccharides and electrolytes during motion could enhance the endurance capability of the human body. Research indicated that the addition of proteins in said beverage could result in an improved effect of saccharide supplementation and facilitate the utilization of saccharides. In particular, the supplementation of a saccharide-beverage comprising proteins after motion was more advantageous for facilitating the resynthesis of glycogens after motion. Moreover, research found that the optimal ratio of saccharides to proteins for achieving the aforementioned purposes was 4:1. However, it has not been found that the supplementation of a saccharide-beverage containing protein could promote the oxidation of fats.

Proteins are not one of the main sources for energy supply during human body motion, and the utilization of proteins further requires a certain amount of time and consumes a lot of energy. Thus, the supplementation of a saccharide-beverage containing proteins during motion of the human body cannot achieve the best effect of instant energy supply. In addition, the supplementation of current saccharide-beverages containing proteins cannot promote fat mobilization and oxidation in vivo.

### DISCLOSURE OF INVENTION

The present invention overcomes the aforementioned drawbacks of current compositions, and provides a composition that is taken up through the gastrointestinal tract before, during and after motion of the human body. In some embodiments of the invention, the composition enhances the endurance capability of the human body during motion, promotes recovery after motion, reduces body fats, and alleviates fatigue of central nerves during motion.

The present invention is implemented by the following technical solutions.

In some embodiments, the present invention provides a composition comprising saccharides and peptides, wherein the peptides are obtained by hydrolyzing proteins. In some embodiments, the ratio of saccharides to peptides in said composition is from 1:1 to 20:1. In particular embodiments of the invention, the ratio of the saccharides to the peptides is from 2:1 to 10:1.

In other embodiments, the composition of the present invention can further comprise vitamins, minerals, flavoring materials, and electrolytes.

In some embodiments, the composition of the present invention is a liquid beverage, while in others it is a solid beverage, a food additive, or an energy bar.

The peptides in the composition of the present invention can be from various sources. In some embodiments of the invention, the peptides are from soybean proteins, while in others they are from wheat proteins, milk proteins, other proteins or mixtures thereof.

In some embodiments of the invention, the saccharides in the composition of the present invention are monosaccharides, while in other embodiments they are disaccharides, polysaccharides (especially oligosaccharides and polysaccharides with middle polymerization degree) or combinations thereof in various proportions. In some embodiments of the invention, the saccharides in the composition of the present invention are edible saccharides, wherein said edible saccharides are sucrose, as well as glucose or maltodextrin.

In some embodiments of the invention, the peptides in the composition are obtained from proteinase hydrolysis. In particular embodiments of the invention, the peptides in the composition of the present invention mainly are short peptides containing 3-6 amino acids. Such short peptides have higher absorption speed and better absorption effects than amino acids and proteins. The absorbed peptides can be directly utilized and take effect more quickly, thereby reducing the metabolic burden and time during the supplementation of proteins. Therefore, the use of a food or beverage comprising the composition of the present invention could bring about better saccharide-supplementation effects than supplementation of saccharides alone, supplementation of saccharides+proteins, or supplementation of saccharides+amino acids. In addition, due to the limitation of the amount of saccharides in the body, the in time mobilization of fats that are abundant with higher energy storage for energy supply during motion has undoubted advantages for enhancing the capability of endurance exercises. Moreover, the supplemented peptides are capable of reducing the decomposition of muscle proteins in the body to generate energy when the energy supply is in short. The reduction of the decomposition of muscle proteins has advantages to protect the muscle. Finally, since peptides can be decomposed in vivo and form partial branched amino acids, the increase of supply of branched amino acids during motion of the human body can elevate the level of branched amino acids in blood, thereby preventing and alleviating fatigue of central nerves.

As compared to the compositions in the prior art, the composition of the present invention has the following beneficial effects: enhancing endurance capability of the human body during motion when a food or beverage containing the composition of the present invention is applied to the human body before, during or after motion; promoting recovery after motion; and reducing body fats and alleviating fatigue of central nerves during motion.

### MODES FOR CARRYING OUT THE INVENTION

The present invention provides a composition comprising saccharides and peptides, that is taken up through the gastrointestinal tract before, during and after motion of the human body. In some embodiments of the invention, the composition enhances the endurance capability of the human body during motion, promotes recovery after motion, reduces body fats, and alleviates fatigue of central nerves during motion. In some embodiments of the invention, the composition is a liquid beverage, while in other embodiments of the invention, the composition is a solid beverage, a food additive, or an energy bar.

In some embodiments of the invention, the saccharides in the composition of the present invention are monosaccharides, while in other embodiments they are disaccharides, polysaccharides (especially oligosaccharides and polysaccharides with middle polymerization degree) or combinations thereof in various proportions. In some embodiments of the invention, the saccharides in the composition of the present invention are edible saccharides, wherein said edible saccharides are sucrose, as well as glucose, maltodextrin or mixtures thereof.

In some embodiments of the invention, the ratio of saccharides to peptides in the composition is from 1:1 to 20:1. In particular embodiments of the invention, the ratio of the saccharides to the peptides is from 2:1 to 10:1.

In some embodiments of the invention, the peptides of the composition are obtained by hydrolyzing proteins. In particular embodiments of the invention, the peptides in the composition are obtained from proteinase hydrolysis. In other particular embodiments of the invention, the peptides mainly are short peptides containing 3-6 amino acids. Such short peptides have higher absorption speed and better absorption effects than amino acids and proteins. The absorbed peptides can be directly utilized and take effect more quickly, thereby reducing the metabolic burden and time during the supplementation of proteins. Therefore, the use of a food or beverage comprising the composition of the present invention could bring about better saccharide-supplementation effects than supplementation of saccharides alone, supplementation of saccharides+proteins, or supplementation of saccharides+amino acids. In addition, due to the limitation of the amount of saccharides in the body, the in time mobilization of fats that are abundant with higher energy storage for energy supply during motion has undoubted advantages for enhancing the capability of endurance exercises. Moreover, the supplemented peptides are capable of reducing the decomposition of muscle proteins in the body to generate energy when the energy supply is in short. The reduction of the decomposition of muscle proteins has advantages to protect the muscle. Finally, since peptides can be decomposed in vivo and form partial branched amino acids, the increase of supply of branched amino acids during motion of the human body can elevate the level of branched amino acids in blood, thereby preventing and alleviating fatigue of central nerves.

The peptides in the composition of the present invention can be from various sources. In some embodiments of the invention, the peptides are from soybean proteins, while in others they are from wheat proteins, milk proteins, other proteins or mixtures thereof. In a preferred embodiment of the invention, the peptides in the composition of the present invention are obtained from soybean proteins.

In some embodiments of the invention, the composition can further comprise vitamins, minerals, flavoring materials, and electrolytes.

As compared to the compositions in the prior art, the composition of the present invention has the following beneficial effects: enhancing the endurance capability of the human body during motion when a food or beverage containing the composition of the present invention is applied to the human body before, during or after motion; promoting recovery after motion; and reducing body fats and alleviating fatigue of central nerves during motion.

The descriptions of the present invention all indicate that the composition of the present invention is capable of enhancing the endurance capability of the human body during motion; promoting recovery after motion; and reducing body fats and alleviating fatigue of central nerves during motion. The present invention is further illustrated as following in combination with the following examples.

### Example 1

### Preparation of a Sports Beverage Comprising a Combination of Saccharides and Peptides

1. The components that should be pre-dissolved, such as sucrose, the powder of peptides, vitamins, etc. . . . were weighed in accordance with a formulation and dissolved with an appropriate amount of water, sufficiently agitated until they were dissolved completely for standby.
2. The pre-dissolved components were poured into a mixing tank, the other components were then added, water was supplemented to achieve the prescribed amount, and they were all mixed by sufficient agitation.
3. The mixed beverage was filtrated, sterilized, filled, packaged, and cooled to finally obtain a finished beverage product.

The sports beverage comprised the following main components (w/w %):

| | |
|---|---|
| Sucrose | 6.5% |
| Soybean peptides | 1.2% |
| Citric acid | 0.14% |
| Sodium citrate | 0.07% |
| Potassium citrate | 0.04% |
| Pectin | 0.04% |
| Sodium chloride | 0.02% |
| Vitamins B, C | 0.01% |
| Defoaming agent | 0.02% |
| Edible essence | 0.09% |
| Water | 91.87% |

### Example 2

### Preparation of a Solid Beverage Comprising a Combination of Saccharides and Peptides

1. According to a formulation, all needed raw materials were weighed for standby;
2. The raw materials were added in order into a solid blender, and were blended homogeneously;
3. The blended materials were weighed and finally packaged on a solid package line to obtain the solid beverage.

The sports beverage comprised the following main components (w/w %):

| | |
|---|---|
| Sucrose | 50% |
| Glucose | 39% |
| Soybean peptides | 5% |
| Vitamins B, C | 0.25% |
| Sodium chloride | 0.8% |
| Potassium citrate | 0.5% |
| Sodium citrate | 1% |
| Citric acid | 1.8% |
| Anti-coagulating agent | 0.65% |
| Edible essence | 1% |

### Example 3

### Effect of a Sports Beverage Comprising a Combination of Saccharides and Peptides

Sports Drink 571 (named Drink 571 hereinafter) comprising a combination of saccharides and peptides was prepared following the method described in example 1. The sports drink comprised the following main components (w/w %):

| | |
|---|---|
| Sucrose | 5.0% |
| Soybean peptide | 1.0% |
| Citric acid | 0.1415% |
| Sodium citrate | 0.07% |
| Potassium Sulphate: | 0.04% |
| Pectin | 0.04% |
| Sodium chloride | 0.02% |
| Vitamin C | 0.005% |
| Vitamin B6 | 0.001% |
| Vitamin B12 | 0.001% |
| Edible essence | 0.09% |
| Water supplemented to | 100% |

Sports Drink 834 (named Drink 834 hereinafter) comprising saccharides, but no added peptides was also prepared following the method as described in example 1. The sports drink comprised the following main components (w/w %):

| | |
|---|---|
| Sucrose | 5.0% |
| Citric acid | 0.1415% |
| Sodium citrate | 0.07% |
| Potassium Sulphate: | 0.04% |
| Pectin | 0.04% |
| Sodium chloride | 0.02% |
| Vitamin C | 0.005% |
| Vitamin B6 | 0.001% |
| Vitamin B12 | 0.001% |
| Edible essence | 0.09% |
| Water supplemented to | 100% |

The following tests were carried out so as to evaluate the effect of Drink 571 and Drink 834 with respect to improvement of the endurance capability of the human body in motion, promotion of recovery after motion, body fat reduction, and alleviation of fatigue of central nerves during motion.

### 1. Test Subjects

12 Professional male athletes consisting of 8 professional cyclists and 4 professional triathletes participated as volunteers in this clinical trial after having signed an “Informed Consent”. These subjects were randomly divided into two groups, and respectively drank Drink 834 and Drink 571 by a double blind method to carry out the predetermined tests. After the end of a first set of tests, all subjects had two weeks of rest, then drank the two drinks crossly, and were subjected to the same tests as before the cross drinking. Since two athletes quit for matches before and after the cross drinking respectively, there were 10 effective cases in the present research.

### 2. Instruments and Devices

Instruments: Cortex 3B cardiopulmonary analyzers; Load power bicycles; IsoMed2000 isokinetic dynamometer; In Body 3.0 Body Composition Analyzer; and other biochemical devices.

### 3. Test Procedure

Pre-tests were conducted on two separate days one week before the actual test began, in order to test the athletes' basic physiological parameters and maximal oxygen uptake (VO₂ₘₐₓ), and to calculate the load corresponding to 70% VO₂ₘₐₓ according to the test results.

Two days before the test, with the aid from nutritionists, the athletes ate a standard diet (55% carbohydrate, 30% fat and 15% protein based on the total energy supply). One day before the test, the athletes stopped training and did not eat after dinner, although they could drink water.

On the day of the test, the athletes first had several minutes to rest in the lab, then they were tested (pre-exercise—“pre-Ex”—) in terms of baseline heart rhythm (mean of three values), biochemical indexes by taking venous blood from the forearm, urine specific gravity (bladder empty) and body composition (including body weight). Ten minutes before the test, the athletes wore a heart rhythm watch, and separately drank the test drinks (6 ml/(kg body weight), once).

After completing the above procedures, the athletes started pedaling at about 10:00-12:00 am. Each of the athletes was tested before and after cross drinking the drinks, and started pedaling at almost the same time with a deviation of no more than 1 h. The whole day tests were completed at about 18:00-20:00.

The athletes pedaled at 150 W for 10 minutes as a warm-up exercise, then pedaled for 110 minutes at a load corresponding to ˜70% of the maximum heart rate (HRmax). During the pedaling, the athletes drank the test drinks at 3 ml/(kg body weight)/15 min. During the pedaling, the athletes' heart rhythms were monitored continuously, and all athletes' rhythms substantially fluctuated around 70% HRmax. After the end of 120 minutes of pedaling, the athletes pedaled at 50 W for 15 minutes for relaxation. Subsequently, the athletes pedaled at 50 W, 150 W and 200 W for 1, 2 and 2 minutes respectively, then pedaled at a load corresponding to ˜85% VO₂ₘₐₓ until they fatigued (the first fatigue—“1stF”—). The criteria for judgment of fatigue was that an athlete could not pedal at 55 rpm for 30 seconds. During exercise, the athletes were asked for subjective feel (Borg Questionnaire). After the exercise (“0 h, 1 stF”), blood samples were taken immediately for testing blood lactic acid and blood sugar, the athletes drank the test drinks at 10 ml/(kg body weight) within 10 minutes after the exercise, urinated (if possible) for measuring the urine quantity and testing the ten indexes of urine, and were tested in terms of body composition.

At the end of 1 hour of exercise (“1 h, 1stF”), blood samples were taken from the forearm for the test of the biochemical indexes, urine was collected and body composition was tested; At the end of 4 hours of exercise (“4 h, 1 stF”), blood samples were taken from the forearm for the test of the biochemical indexes. Urine was collected and body composition was also tested. Then, the second fatigue test—“2ndF”—(pedaling until fatigued) was conducted. The pedaling procedure was essentially identical to that for the first fatigue test. The athletes first pedaled at 50 W, 150 W and 200 W separately for 6 minutes, 2 minutes and 2 minutes, then pedaled at a load corresponding to ˜85% VO₂ₘₐₓ until they fatigued, and the indexes used for the first fatigue test were chosen for the test. Then, the athletes were administered the test drinks at 10 ml/kg twice separately at 8 and 12 hours after the first fatigue test.

Twenty four hours after the exercise (“24 h, 1 stF”), blood was collected and the ten indexes of urine were tested. Then, the athletes drank the test drinks at 6 ml/kg. The athletes were administered the test drinks at 10 ml/kg when they left the lab. Forty-eight hours after the exercise (“48 h, 1 stF”), blood samples were taken from the forearm for testing of the biochemical indexes.

After the implementation of the first test, the athletes rested for 15 days, drank the test drinks crossly, and were subjected to the tests with the same procedure.

### 4. Test Results

### 1) Effect on Improvement of Endurance Capability of the Human Body in Motion by Taking Drink 571 and Drink 834

In the first fatigue test, the fatigue time of the 571 group was slightly longer than that of the 834 group (5.1 minutes longer, up to 21.3%). The results were shown in Table 1.

TABLE 1
time to exhaustion (min)

| Group | 1st F (n = 10) |
|---|---|
| 571 | 29.0 ± 3.5 |
| 834 | 23.9 ± 2.5 |

Conclusion: aerobic endurance was enhanced to some extent by drinking Drink 571.

2) Effect on Improvement of Promotion of Recovery after Motion, by Taking Drink 571

### i) Assay of Urine Protein Content in Urine Examination

For comparing urine examinations, the qualitative results of urine protein (PROT) were scored: 1 for “trace”, 2 for “+”, 3 for “++”, and the like. According to table 2, from the beginning of the exercise to 4 h after the first fatigue test, the total urinary output of the 571 group was slightly lower than that of the 834 group (11%). During the test, the score of urine protein of the 571 group was lower than that of the 834 group. The results are shown in Table 2.

TABLE 2
urine protein content in urine examination

| | |
|---|---|
| volume (ml) | protein |

| Time | 571 | 834 | 571 | 834 |
|---|---|---|---|---|
| Pre-Ex | — | — | 5 | 2 |
| 0 h, 1stF | 306.4 | 281.1 | 4 | 11 |
| 1 h, 1stF | 50.4 | 71.1 | 12 | 14 |
| 4 h, 1stF | 243.0 | 314.6 | 3 | 7 |
| Sub-total | 599.8 | 666.8 | 24.0 | 34.0 |
| 0 h, 2ndF | 119.0 | 57.4 | 4 | 20 |
| 1 h, 2ndF | 68.3 | 40.1 | 2 | 8 |
| Total | 787 | 764 | 30.0 | 62.0 |

### ii) Change of Myoglobin Content in Blood

Results of changes in myoglobin content in blood during the test are shown in Table 3.

TABLE 3
Change of myoglobin content in blood (n = 10)

| parameter | group | Pre-Ex | 0 h, 1stF | 4 h, 1stF | 24 h, 1stF | 48 h, 1stF |
|---|---|---|---|---|---|---|
| myoglobin | 571 | 64.5 ± 2.8 | 181.1 ± 34.4$ | 118.3 ± 18.6$ | 76.8 ± 5.1$ | 66.8 ± 3.2 |
| (μg/L) | 834 | 66.3 ± 2.3 | 208.9 ± 60.6& | 134.8 ± 29& | 98.1 ± 12.4& | 66.1 ± 3.5 |

*p < 0.05 compared with 834;
#0.05 < P < 0.1 compared with 834;
$P < 0.05 compared with Pre-Ex of the same group;
&0.05 < P < 0.1 compared with Pre-Ex of the same group.

As indicated by the results of changes in urine protein shown in Table 2 and myoglobin content shown in Table 3, the drinking of Drink 571 could reduce the discharge of urine proteins and protect muscle cellular membrane from damage in order to reduce the release of myoglobin to blood. In the meantime, it could expedite the recovery of damaged muscle cellular membrane during exercise.

Conclusion: The drinking of Drink 571 could alleviate muscle damage and/or expedited recovery of muscle damage.

### 2) Effect on Body Fat Reduction by Taking Drink 571 and Drink 834

The fat percentage was tested prior to exercise, during exercise and after exercise and the results are shown in Table 4.

TABLE 4
Result of fat percentage as tested prior to exercise,
during exercise and after exercise

| | | Body weight | Body fat | Body fat % |
|---|---|---|---|---|
| group | time | (kg) | (kg) | (%) |
| 571 | Pre-Ex | 73.7 ± 3.2 | 8.7 ± 0.6* | 11.9 ± 0.7* |
| (n = 10) | 0 h, 1stF | 72.5 ± 3.3$ | 7.0 ± 0.6$ | 9.7 ± 0.8$ |
| | 1 h, 1stF | 72.4 ± 3.2 | 7.1 ± 0.6 | 9.9 ± 0.8 |
| | 4 h, 1stF | 73.1 ± 3.2$ | 7.8 ± 0.8$ | 10.7 ± 1.0$ |
| Change between 4 h, | | −0.6 | −0.9 | −1.2 |
| 1st F and Pre-Ex | | | | |
| 834 | Pre-Ex | 73.6 ± 3.1 | 8.3 ± 0.6 | 11.3 ± 0.8 |
| (n = 10) | 0 h, 1stF | 72.4 ± 3.2$ | 7.0 ± 0.6$ | 9.8 ± 0.9$ |
| | 1 h, 1stF | 72.4 ± 3.1 | 7.3 ± 0.6 | 10.2 ± 0.9 |
| | 4 h, 1stF | 73.0 ± 3.2 | 7.5 ± 0.7 | 10.4 ± 0.9 |

| | | | |
|---|---|---|---|
| Change between 4 h, | −0.6 | −0.8 | −0.9 |
| 1st F and Pre-Ex | | | |

*p < 0.05 compared with 834;
$P < 0.05 compared with Pre-Ex of the same group

The results of the test for body composition indicated that after the athletes of the two groups firstly pedaled to fatigue, their mean body weight decreased by 1.2 kg, and even after 4 h of recovery, their mean body weight still decreased by 0.6 kg. With regards to the fat weight and fat percentage before exercise, the 571 group was significantly higher than 834 group; after exercise, a decrease occurred in both groups, but the 571 group had more decrease.

Conclusion: The effect on body fat reduction by drinking Drink 571 is better than that of Drink 834.

### 4) Effect on Alleviation of Fatigue of Central Nerves During Motion by Drinking Drink 571 and Drink 834

The following tests were taken when evaluating the effect on alleviation of fatigue of central nerves during motion by drinking Drink 571 and Drink 834.

### i) Rating of Perceived Exertion

Perceived exertions were recorded for athletes when they pedaled for 2 h at 70% HRmax and during the first fatigue test. At the end of 2 h of pedaling and during the first fatigue test, the athletes of the 834 group fatigued more than the athletes of the 571 group.

ii) Results of the changing amount of branch amino acids as well as the ratio of branch chain amino acids to aromatic amino acids (BCAA/AAA ratio) in blood as measured at the time prior to exercise, during exercise and after exercise. The results are shown in Table 5.

TABLE 5
Changing amount of branch amino acids as well
as the ratio of branch chain amino acids to aromatic
amino acids (BCAA/AAA ratio) in blood

| | | BCAA | | AAA | | BCAA/AAA | |
|---|---|---|---|---|---|---|---|
| time | | 571 | 834 | 571 | 834 | 571 | 834 |
| Pre-Ex | Mean | 0.572 | 0.580 | 0.184 | 0.194 | 3.116 | 3.050 |
| | SE | 0.043 | 0.045 | 0.012 | 0.017 | 0.12 | 0.15 |
| 0 h, 1st F | Mean | 0.549 | 0.504 | 0.209 | 0.199 | 2.684 | 2.509 |
| | SE | 0.042 | 0.057 | 0.017 | 0.016 | 0.16 | 0.16 |
| 4 h, 1st F | Mean | 0.769 | 0.647 | 0.248 | 0.246 | 3.107 | 2.667 |
| | SE | 0.068 | 0.063 | 0.016 | 0.021 | 0.19 | 0.16 |
| 24 h, 1st F | Mean | 0.587 | 0.579 | 0.198 | 0.205 | 2.986 | 2.854 |
| | SE | 0.035 | 0.043 | 0.012 | 0.015 | 0.09 | 0.11 |
| 48 h, 1st F | Mean | 0.618 | 0.614 | 0.212 | 0.207 | 2.969 | 2.947 |
| | SE | 0.037 | 0.065 | 0.017 | 0.019 | 0.08 | 0.133 |

Conclusion: directly and indirectly reflecting the fatigue degree of central nervous system included: subjective feel (Borg Questionnaire), serum amino acids content, BCAA/AAA, etc. . . . In the trial, the 834 group felt more fatigue at the end of 2 h pedaling and during the first fatigue test than the 571 group. Both the BCAA content and the ratio of BCAA to AAA of the 571 group indicated that the Drink 571 might play an important role for alleviating and/or preventing central nervous system fatigue.

In general, the optimal embodiment of the present invention is: drinking a small amount of the beverage before exercising and drinking regularly during exercise. This can increase the sources of energy substances and effectively save the limited high-grade energy source—saccharide. It can also increase the content of branched amino acids in the meantime, improve heart and lung function, alleviate body and central nerve fatigue, alleviate muscle damage before and during exercise, as well as expedite the recovery of impaired muscle by drinking the beverage in time after exercise.

All descriptions and examples in the invention are for references. The present invention is illustrated in detail in the examples. All examples are improvable in technology as long as the main concept of the present invention is complied with.

1. A composition comprising saccharides and peptides, wherein said peptides are obtained by hydrolyzing proteins, characterized in that the ratio of the saccharides to the peptides in said composition is from 1:1 to 20:1.

2. The composition according to claim 1, characterized in that the peptides are derived from soybean proteins, wheat proteins, corn proteins, pea proteins, or mixtures thereof.

3. The composition according to claim 1, characterized in that the peptides have an average peptide chain length of 3 to 20 amino acids.

4. The composition according to claim 1, characterized in that the peptides have an average peptide chain length of 3 to 6 amino acids.

5. The composition according to claim 1, characterized in that the saccharides are monosaccharides, disaccharides, polysaccharides, or mixtures thereof.

6. The composition according to claim 1, characterized in that the saccharides are sucrose, glucose, maltodextrin, or mixtures thereof.

7. The composition according to claim 1, characterized in that the saccharides are sucrose.

8. The composition according to claim 1, characterized in that the ratio of the saccharides to the peptides is from 2:1 to 10:1.

9. The composition according to claim 1 or 8, characterized in that said composition further comprises at least one component selected from the group consisting of: vitamins, flavoring materials, electrolytes and minerals.

10. The composition according to claim 1 or 8, characterized in that said composition is a liquid beverage, a solid beverage, a food additive or an energy bar.

11. A method for enhancing endurance capability during human body motion, characterized in that the composition according to claim 1 or 8 is supplemented through the gastrointestinal tract before, during or after human body motion.

12. A method for promoting recovery after human motion, characterized in that the composition according to claim 1 or 8 is supplemented through the gastrointestinal tract before, during or after human body motion.

13. A method for promoting body fat reduction during human body motion, characterized in that the composition according to claim 1 or 8 is supplemented through the gastrointestinal tract before, during or after human body motion.

14. A method for alleviating fatigue of central nerves during human motion, characterized in that the composition according to claim 1 or 8 is supplemented through the gastrointestinal tract before, during or after human body motion.
